(19) United States
(12) Patent Application Publication
(10) Pub. No.: US 2013/0203185 A1
(43) Pub. Date: Aug. 8, 2013

(54) METHOD FOR IMMOBILIZING A PROTEIN ON SELF-ASSEMBLED MONOLAYER

(71) Applicant: Panasonic Corporation, Osaka (JP)

(72) Inventor: Yukari HATAOKA, Kyoto (JP)

(73) Assignee: PANASONIC CORPORATION, Osaka (JP)

(21) Appl. No.: 13/829,506

(22) Filed: Mar. 14, 2013

Related U.S. Application Data
(63) Continuation of application No. PCT/JP2011/007239, filed on Dec. 22, 2011.

(30) Foreign Application Priority Data
Jul. 8, 2011 (JP) 2011-151573

(51) Int. Cl. C07K 1/107

(52) U.S. Cl. CPC C07K 1/1075
USPC 436/501; 530/409

(57) ABSTRACT

One molecule of the amino acid selected from the five kinds of amino acids consisting of cysteine, lysine, histidine, phenylalanine, and glycine is interposed between a self-assembled monolayer and a molecule of a protein. A method for immobilizing an protein on a self-assembled monolayer includes the following steps (a) and (b) in this order: a step (a) of preparing a substrate including one molecule of an amino acid and the self-assembled monolayer and a step (b) of supplying the protein to the substrate to form a peptide bond represented by a predetermined chemical formula as a result of reaction between the carboxyl group of the one molecular of the amino acid and the amino group of the protein.

### CROSS-REFERENCE TO RELATED APPLICATIONS

This is a continuation of International application No. PCT/JP2011/007239, with international filling date of Dec. 22, 2011, which claims priority of Japanese Patent Application No. 2011-151573, filed on Jul. 8, 2011, the contents of all of which are hereby incorporated by reference.

Further, it is noted that International Patent Publication Nos. WO2011/089903, WO2012/029202, WO2012/053138, WO2012/168988 and WO2013/005269 are commonly owned by the Assignee of the present application.

### BACKGROUND

The present disclosure relates to a method for immobilizing a protein on a self-assembled monolayer.

A biosensor is used to detect or quantify a target substance contained in a sample. Some biosensors include protein capable of binding to the target substance to detect or quantify the target substance. More particularly, a biosensor for detecting or quantifying an antigen includes an antibody capable for binding specifically to the antigen. Similarly, biosensors for detecting or quantifying biotin and glucose include streptavidin and glucose oxidase, respectively.

When a sample containing the target substance is supplied to the biosensor including protein capable of binding to the target substance, the target substance is bound to the protein to detect or quantify the target substance.

International Patent Publication No. WO00/04382 discloses a conventional biosensor including protein. WO00/04382 corresponds to Japanese Publication of a translation of PCT international application No. 2002-520618 (see, e.g. Page 24, lines 23-26, Page 25, lines 3-20, Page 25, line 27-Page 26, line 13, Page 26, lines 14-22, Page 28, lines 21-23, Page 32, lines 3-29, Page 35, and line 21-Page 36, line 21 of WO00/04382 or paragraphs [0080], [0082], [0084], [0085], [0095], [0109], [0118], and [0119] of the corresponding Japanese Publication). FIG. 2 shows a biosensor disclosed in FIG. 7 of Patent Literature 1.

According to the description regarding FIG. 7 of WO00/04382, the biosensor is used for screening an activity of a biomolecule. The biosensor includes a monolayer 7, an affinity tag 8, an adaptor molecule 9, and a protein 10. The monolayer 7 is composed of a self-assembled monolayer represented by chemical formula: X—R—Y (see, Page 24, lines 23-26, Page 25, lines 3-20, Page 25, line 27-Page 26, line 13, and Page 26, lines 14-22 of WO00/04382; or paragraphs [0080], [0082], [0084] and [0085] of the corresponding Japanese Publication). Examples of X, R, and Y are HS—, an alkane, and a carboxyl group, respectively (see, Page 25, lines 3-20, Page 25, lines 27-Page 26, line 13, and Page 28, lines 21-23 of WO00/04382; or paragraphs [0084], [0085], and [0095] of the corresponding Japanese Publication).

### BRIEF SUMMARY

### Technical Problem

In order to improve the detection sensitivity or the quantification accuracy of the target substance, it is required to increase an amount of protein to be immobilized on the biosensor.

The present inventor has discovered that the amount of the immobilized protein per unit area was increased significantly by binding one molecule amino acid selected from the group consisting of cysteine, lysine, histidine, phenylalanine, and glycine to a self-assembled monolayer and then immobilizing protein. The present subject matter has been provided on the basis of the discovery.

The purpose of the present disclosure is to provide a method for increasing an amount of protein to be immobilized on the self-assembled monolayer, and a sensor with the protein immobilized in accordance with the same method.

### Solution to Problem

A method for immobilizing a protein on a self-assembled monolayer includes the following steps. Step (a) is a step of preparing a substrate including one molecule of an amino acid and the self-assembled monolayer. The one molecule of the amino acid is bound to the self-assembled monolayer through a peptide bond represented by the following chemical formula (I):

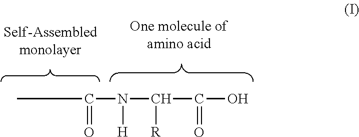

where R represents the side chain of one molecule of the amino acid. The one molecular of the amino acid is selected from the five kinds of amino acids consisting of cysteine, lysine, histidine, phenylalanine, and glycine. Step (b) is a step of supplying the protein to the substrate to form a peptide bond represented by the following chemical formula (II) as a result of reaction between the carboxyl group of the one molecule of the amino acid and the amino group of the protein:

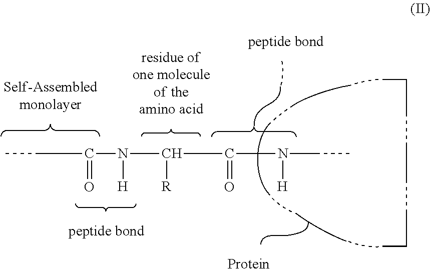

where R represents the side chain of the one molecule of the amino acid.

In one embodiment, the step (a) may include the following steps (a1) and (a2). Step (a1) is a step of preparing the substrate comprising the self-assembled monolayer on the surface thereof, the self-assembled monolayer having a carboxyl acid at one end. Step (a2) is a step of supplying the one molecule of the amino acid to form the peptide bond represented by the chemical formula (I) as a result of reaction between the carboxyl group of the one end of the self-assembled monolayer and the amino group of the one molecule of the amino acid.

In one embodiment, the method may further include, between the step (a) and the step (b), a step (ab) of activating the carboxyl group of the one molecule of the amino acid with a mixture of N-Hydroxysuccinimide and 1-ethyl-3-(3-dimethylaminopropyl) carbodiimide hydrochloride.

In one embodiment, the method may further includes, between the step (a1) and the step (a2), a step (a1a) of activating the carboxyl group of the self-assembled monolayer with a mixture of N-Hydroxysuccinimide and 1-ethyl-3-(3-dimethylaminopropyl)carbodiimide hydrochloride.

In one embodiment, the chemical formula (II) may be represented by the following chemical formula (III):

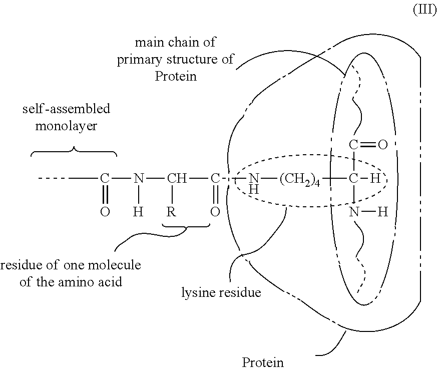

where R represents the side chain of the one molecule of the amino acid.

One aspect of the present disclosure is a sensor including a self-assembled monolayer, one molecule of an amino acid, and a protein. The one molecule of the amino acid is interposed between the self-assembled monolayer and the protein, and the protein is bound to the self-assembled monolayer through two peptide bonds represented by the following chemical formula (II):

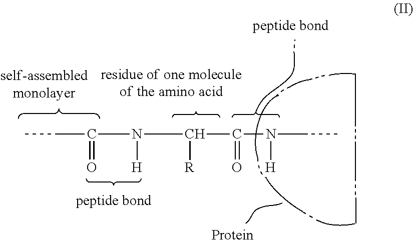

where R represents the side chain of the one molecule of the amino acid. The one molecule of the amino acid is selected from the five kinds of amino acids consisting of cysteine, lysine, histidine, phenylalanine, and glycine.

In one embodiment, the chemical formula (II) may be represented by the following chemical formula (III):

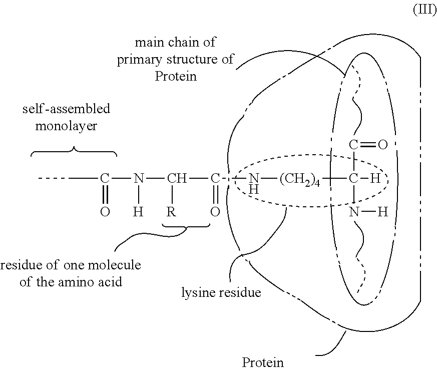

where R represents the side chain of the one molecule of the amino acid.

One aspect of the present disclosure is a method for detecting or quantifying a target substance contained in a sample with use of a sensor. The method includes the following steps. Step (a) is a step of preparing the sensor including a self-assembled monolayer, one molecule of an amino acid, and a protein. The one molecule of the amino acid is interposed between the self-assembled monolayer and the protein, and the protein is bound to the self-assembled monolayer through two peptide bonds represented by the following chemical formula (II):

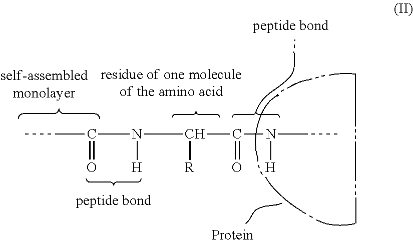

where R represents the side chain of the one molecule of the amino acid. The one molecule of the amino acid is selected from the five kinds of amino acids consisting of cysteine, lysine, histidine, phenylalanine, and glycine. Step (b) is a step of supplying the sample to the sensor to bind the target substance to the protein. Step (c) is a step of detecting the target substance bound in the step (b), or quantifying the target substance contained in the sample from the amount of the target substance bound in the step (b).

In one embodiment, the chemical formula (II) may be represented by the following chemical formula (III):

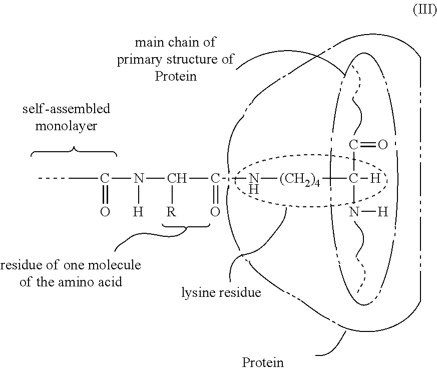

where R represents the side chain of the one molecule of the amino acid.

### Advantageous Effect

The present subject matter can increase significantly the amount of the protein to be immobilized per unit area.

### BRIEF DESCRIPTION OF THE DRAWINGS

FIG. 1 shows an exemplary schematic view of a method according to one embodiment of the present disclosure.

FIG. 2 corresponds to FIG. 7 of WO00/04382.

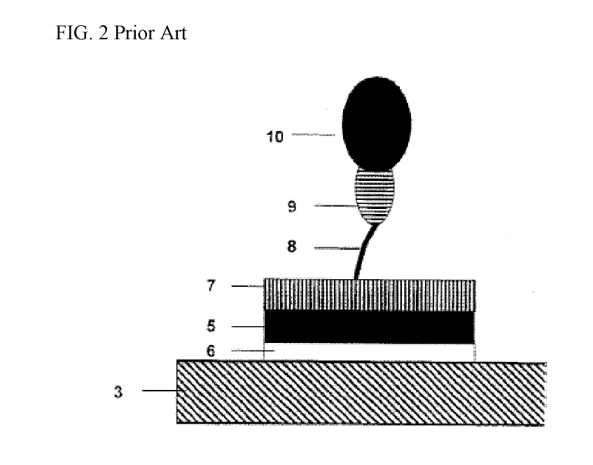

FIG. 3 shows a schematic view of a method according to the prior art.

### DETAILED DESCRIPTION

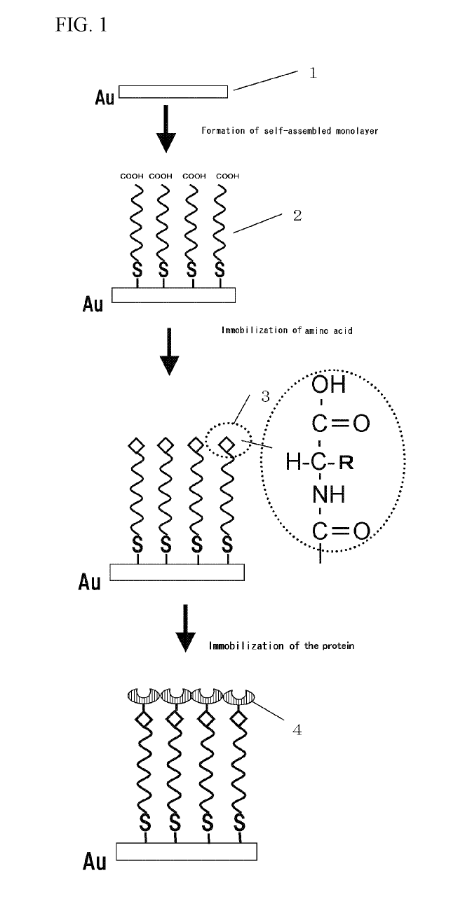

The embodiment of the present disclosure is described below with reference to FIG. 1.

FIG. 1 shows an exemplary method according to the present disclosure for immobilizing protein on a self-assembled monolayer.

Preferably, a substrate 1 is a gold substrate. An example of the gold substrate is a substrate having gold uniformly on its surface. Specifically, the gold substrate may be a substrate having a gold film formed by a sputtering method on the surface of glass, plastic, or SiO₂.

First, the substrate 1 is immersed into a solvent containing an alkanethiol. Preferably, the substrate is washed before immersed. The alkanethiol has a carboxyl group at the end thereof. It is preferable that the alkanethiol has the carbon number within the range from six to eighteen. Thus, a self-assembled monolayer 2 is formed on the substrate 1.

The preferred concentration of the alkanethiol is approximately 1 mM to 10 mM. The solvent is not limited to, as long as it dissolves the alkanethiol. An example of the preferred solvent is ethanol, dimethyl sulfoxide (hereinafter, referred to as “DMSO”), and dioxane. The preferred immersing period is approximately 12 to 48 hours.

Next, an amino acid 3 is supplied to the self-assembled monolayer 2. The carboxyl group (—COOH), which is located at the top end of the self-assembled monolayer 2, reacts with an amino group (—NH₂) of the amino acid 3 to form a peptide bond represented by the following the chemical formula (I):

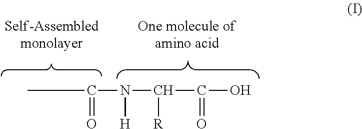

where R represents the side chain of the one molecule of the amino acid.

In the chemical formula (I), one molecule of the amino acid 3 binds to the self-assembled monolayer 2.

The amino acid 3 is selected from five kinds of amino acids consisting of cysteine, lysine, histidine, phenylalanine, and glycine. In other words, in the chemical formula (I), R is the side chain of these five kinds of amino acids.

When the amino acid 3 is supplied to the self-assembled monolayer 2, two or more kinds of amino acids may be supplied simultaneously. In other words, when a solution containing the amino acid 3 is supplied to the self-assembled monolayer 2, the solution may contain two or more kinds of the amino acids 3. In light of uniform bind of the protein to the amino acid 3, which is described later, it is preferred that the solution contains a sole kind of amino acid.

Subsequently, protein 4 is supplied. The 5′-terminal amino group of the protein 4 reacts with the carboxyl group of the amino acid 3. The amino group of the lysine included in the protein also reacts with the carboxyl group of the amino acid 3. Thus, two peptide bonds represented by the following chemical formula (II) are formed to obtain a sensor:

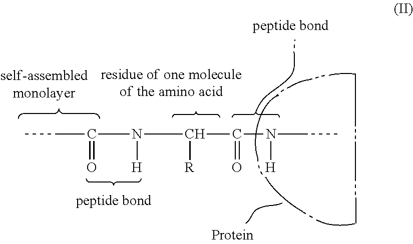

where R represents the side chain of the one molecule of the amino acid.

One molecule of the protein 4 has only one N-terminus (the start of the protein terminated by an amino acid with a free amine group), corresponding to the 5′ end of mRNA encoding the protein, whereas the one molecule of the protein 4 has a lot of lysine groups having a free amine group. Therefore, almost all of the chemical formula (II) is represented more specifically by the following chemical formula (III):

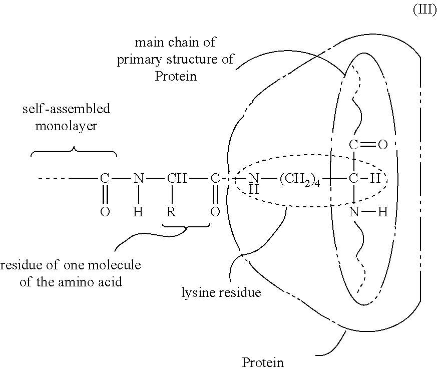

where R represents the side chain of the one molecule of the amino acid.

The obtained sensor is used for detecting or quantifying the target substance contained in the sample.

### EXAMPLES

The following examples and comparative examples describe the present subject matter in more detail.

### Comparative Example A 1

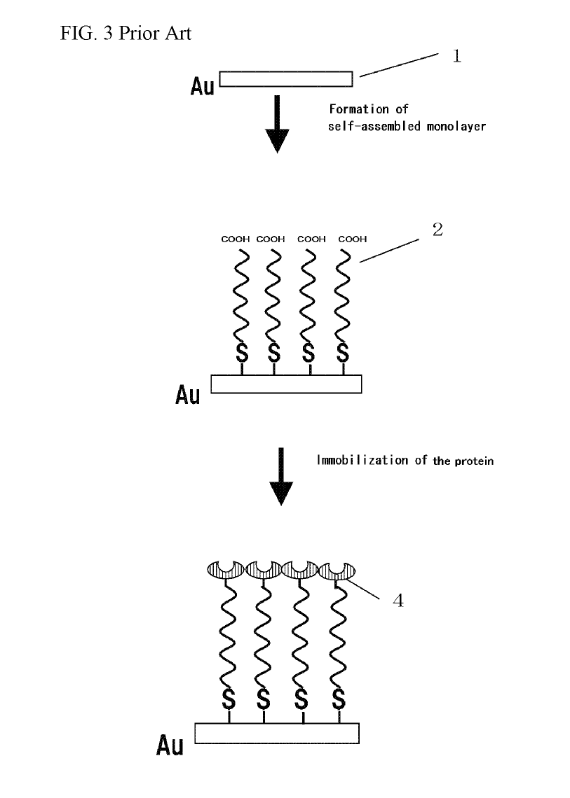

As shown in FIG. 3, Protein A was bound directly with an amide coupling reaction to a carboxyl group located at the top end of self-assembled alkanethiol formed on the gold surface to immobilize the Protein A. The procedure and the results were described below. It is well-known that Protein A is a protein which constitutes five percent of the cell wall of staphylococcus aureus and is abbreviated as “SpA”.

### [Preparation of a Sample Solution]

A sample solution of 16-Mercaptohexadecanoic acid with final concentration of 10 mM was prepared. The solvent thereof was ethanol.

### [Formation of a Self-Assembled Monolayer]

A gold substrate (available from GE healthcare company, BR-1004-05) with gold vapor-deposited on glass was used as a substrate 1. The substrate 1 was washed for ten minutes with a piranha solution containing concentrated sulfuric acid and 30% hydrogen peroxide water. The volume ratio of the concentrated sulfuric acid to the 30% hydrogen peroxide water contained in the piranha solution was 3:1.

Subsequently, the gold substrate was immersed in the sample solution for 18 hours to form a self-assembled monolayer on the surface of the gold substrate. Finally, the substrate 1 was washed with pure water and dried.

### [Immobilization of Protein]

As protein, Protein A was bound to the carboxyl acid group located at the top end of the 16-Mercaptohexadecanoic acid which formed the self-assembled monolayer to immobilize the Protein A.

Specifically, the carboxyl acid group located at the top end of the 16-Mercaptohexadecanoic acid was activated with use of 35 microliters of a mixture of 0.1M NHS (N-Hydroxysuccinimide) and 0.4M EDC (1-ethyl-3-(3-dimethylaminopropyl) carbodiimide hydrochloride). Subsequently, 35 microliters of the Protein A (40 ug/ml) was added at the flow rate of 5 microliters/minute. Thus, the carboxyl acid of the 16-Mercaptohexadecanoic acid was coupled with the amino group of the Protein A.

### Example A1

Experiment was conducted similarly to the comparative example A1 except that glycine was supplied as the one molecule of the amino acid between the formation of the self-assembled monolayer and the immobilization of the Protein A. The procedure and the results are described below.

### [Immobilization of Amino Acid (Glycine)]

Glycine was bound with the carboxyl group located at the top end of the 16-Mercaptohexadecanoic acid which formed the self-assembled monolayer 2 to immobilize the glycine.

Specifically, after the carboxyl group was activated similarly to the comparative example A1, 35 microliters of 0.1M glycine (pH: 8.9) was added at the flow rate of 5 microliters/minute. Thus, the carboxyl group of 16-Mercaptohexadecanoic acid was coupled with the amino group of the glycine.

### [Immobilization of Protein]

Subsequently, Protein A was bound to the carboxyl group of the glycine to immobilize the Protein A. Specifically, after the carboxyl group of the glycine was activated similarly to the above, 35 microliters of Protein A (concentration: 250 micrograms/ml) was added at the flow rate of 5 microliters/minute. Thus, the carboxyl group was coupled with the 5′-terminal amino acid of the Protein A or the amino group of the lysine included in the Protein A.

### [Comparison of the Immobilization Amounts]

The immobilization amounts in the example A1 and in the comparative example A1 were measured with use of an SPR device, Biacore 3000 (available from GE healthcare company).

The term “immobilization amount” means the amount of the protein immobilized per unit area.

### Comparative Examples A2-A16

Serine, alanine, glutaminic acid, methionine, leucine, valine, threonine, isoleucine, tyrosine, asparagine, tryptophan, aspartic acid, arginine, proline and glutamine were used instead of glycine, and each immobilization amount was measured similarly to the case of the example A1.

### Examples A2-A5

Cysteine, lysine, histidine and phenylalanine were used instead of glycine, and each immobilization amount was measured similarly to the case of the example A1.

Table 1 shows the immobilization amounts of Protein A in accordance with the examples A1-A5 and the comparative examples A1-A16.

TABLE 1

| | | |
|---|---|---|
| Example A2 | Cysteine | 17.96117 |
| Example A3 | Lysine | 14.27184 |
| Example A4 | Histidine | 11.35922 |
| Example A5 | Phenylalanine | 10.87379 |
| Example A1 | Glycine | 9.708738 |
| Comparative Example A16 | Asparagine | 9.223301 |
| Comparative Example A2 | Methionine | 9.126214 |
| Comparative Example A3 | Serine | 8.932039 |
| Comparative Example A4 | Tyrosine | 6.850394 |
| Comparative Example A5 | Tryptophan | 8.349515 |
| Comparative Example A6 | Leucine | 7.76699 |
| Comparative Example A7 | Glutamine | 7.378641 |
| Comparative Example A8 | Alanine | 7.281553 |
| Comparative Example A9 | Isoleucine | 5.533981 |
| Comparative Example A10 | Threonine | 5.242718 |
| Comparative Example A11 | Proline | 4.07767 |
| Comparative Example A12 | Glutamic acid | 3.203883 |
| Comparative Example A13 | Aspartic acid | 2.427184 |
| Comparative Example A14 | Valine | 2.106796 |
| Comparative Example A15 | Argnine | 0.621359 |
| Comparative Example A1 | (None) | 1 |

### Examples B1-B5 and Comparative examples B1-B16

Experiments similar to the example A1-A5 and the comparative examples A1-A16 were conducted except that streptavidin was used instead of Protein A.

Table 2 shows the immobilization amounts of the streptavidin in accordance with the examples B1-B5 and the comparative examples B1-B16.

TABLE 2

| | | |
|---|---|---|
| Example B2 | Lysine | 33 |
| Example B3 | Histidine | 32.2 |
| Example B4 | Phenylalanine | 28.8 |
| Example B5 | Cysteine | 26.9 |
| Example B1 | Glycine | 25.6 |
| Comparative Example B16 | Methionine | 25.6 |
| Comparative Example B2 | Glutamic acid | 24.2 |
| Comparative Example B3 | Tyrosine | 24.1 |
| Comparative Example B4 | Alanine | 21.8 |
| Comparative Example B5 | Serine | 20.5 |
| Comparative Example B6 | Aspartic acid | 19.7 |
| Comparative Example B7 | Asparagine | 18.6 |
| Comparative Example B8 | Leucine | 12.9 |
| Comparative Example B9 | Tryptophan | 12 |
| Comparative Example B10 | Threonine | 9.1 |
| Comparative Example B11 | Isoleucine | 6.4 |
| Comparative Example B12 | Valine | 6.1 |
| Comparative Example B13 | Glutamine | 3.6 |
| Comparative Example B14 | Proline | 3.1 |
| Comparative Example B15 | Argnine | 2.5 |
| Comparative Example B1 | (None) | 1 |

### Examples C1-C5 and Comparative examples C1-C16

Experiments similar to the example A1-A5 and the comparative examples A1-A16 were conducted except that glucose oxidase was used instead of Protein A.

Table 3 shows the immobilization amounts of the glucose oxidase in accordance with the examples C1-C5 and the comparative examples C1-C16.

TABLE 3

| | | |
|---|---|---|
| Example C2 | Cysteine | 37.69685 |
| Example C3 | Lysine | 36.59207 |
| Example C4 | Histidine | 36.16066 |
| Example C5 | Phenylalanine | 30.35305 |
| Example C1 | Glycine | 30.32874 |
| Comparative Example C16 | Methionine | 29.62198 |
| Comparative Example C2 | Serine | 29.40409 |
| Comparative Example C3 | Alanine | 26.89383 |
| Comparative Example C4 | Asparagine | 25.171 |
| Comparative Example C5 | Leucine | 23.02633 |
| Comparative Example C6 | Tyrosine | 22.1215 |
| Comparative Example C7 | Glutamic acid | 20.36339 |
| Comparative Example C8 | Isoleucine | 17.82311 |
| Comparative Example C9 | Threonine | 15.35175 |
| Comparative Example C10 | Aspartic acid | 14.48565 |
| Comparative Example C11 | Tryptophan | 12.91537 |
| Comparative Example C12 | Valine | 10.40278 |
| Comparative Example C13 | Argnine | 6.055117 |
| Comparative Example C14 | Proline | 5.792629 |
| Comparative Example C15 | Glutamine | 1.202646 |
| Comparative Example C1 | (None) | 1 |

### Examples D1-D5 and Comparative Examples D1-D16

Experiments similar to the example A1-A5 and the comparative examples A1-A16 were conducted except that antibody was used instead of Protein A.

Table 4 shows the immobilization amounts of the antibody in accordance with the examples D1-D5 and the comparative examples D1-D16.

TABLE 4

| | | |
|---|---|---|
| Example D2 | Histidine | 23.86045 |
| Example D3 | Cysteine | 22.74856 |
| Example D4 | Lysine | 20.91865 |
| Example D5 | Phenylalanine | 18.86891 |
| Example D1 | Glycine | 18.63296 |
| Comparative Example D16 | Tryptophan | 17.46708 |
| Comparative Example D2 | Methionine | 16.50562 |
| Comparative Example D3 | Serine | 16.01948 |
| Comparative Example D4 | Asparagine | 15.96672 |
| Comparative Example D5 | Tyrosine | 15.85254 |
| Comparative Example D6 | Alanine | 15.40134 |
| Comparative Example D7 | Glutamic acid | 14.41335 |
| Comparative Example D8 | Threonine | 13.00732 |
| Comparative Example D9 | Leucine | 8.816629 |
| Comparative Example D10 | Valine | 5.974514 |
| Comparative Example D11 | Isoleucine | 5.701262 |
| Comparative Example D12 | Aspartic acid | 3.676188 |
| Comparative Example D13 | Proline | 3.276342 |
| Comparative Example D14 | Argnine | 2.457678 |
| Comparative Example D15 | Glutamine | 1.171725 |
| Comparative Example D1 | (None) | 1 |

### Examples E1-E5 and Comparative examples E1-E16

Experiments similar to the example A1-A5 and the comparative examples A1-A16 were conducted except that albumin was used instead of Protein A.

Table 5 shows the immobilization amounts of the antibody in accordance with the examples E1-E5 and the comparative examples E1-E16.

TABLE 5

| | | |
|---|---|---|
| Example E2 | Cysteine | 19.49204 |
| Example E3 | Lysine | 18.39829 |
| Example E4 | Histidine | 16.81413 |
| Example E5 | Phenylalanine | 15.16347 |
| Example E1 | Glycine | 14.39286 |
| Comparative Example E16 | Serine | 12.94221 |
| Comparative Example E2 | Alanine | 12.7583 |
| Comparative Example E3 | Glutamic acid | 11.42908 |
| Comparative Example E4 | Methionine | 11.05119 |
| Comparative Example E5 | Leucine | 10.66873 |
| Comparative Example E6 | Valine | 8.958131 |
| Comparative Example E7 | Threonine | 8.8923 |
| Comparative Example E8 | Isoleucine | 8.802846 |
| Comparative Example E9 | Tyrosine | 8.288947 |
| Comparative Example E10 | Asparagine | 8.018876 |
| Comparative Example E11 | Tryptophan | 7.88124 |
| Comparative Example E12 | Aspartic acid | 6.962646 |
| Comparative Example E13 | Argnine | 5.856666 |
| Comparative Example E14 | Proline | 3.829463 |
| Comparative Example E15 | Glutamine | 3.654396 |
| Comparative Example E1 | (None) | 1 |

A skilled person would understand the followings from Table 1 to Table 5.

When the one molecule of the amino acid selected from the five kinds of amino acids consisting of cysteine, lysine, histidine, phenylalanine and glycine is interposed between the self-assembled monolayer and the protein, the immobilization amount of the protein per unit area is increased, compared to the case where the one molecule of the amino acid selected from other fifteen kinds of the amino acid is used or to the case where one molecule of the amino acid is not used.

### INDUSTRIAL APPLICABILITY

The present subject matter can increase significantly the amount of the protein to be immobilized per unit area. This improves the sensitivity or the accuracy of the biosensor. The biosensor may be used for an inspection or a diagnosis which requires the detection or the quantification of the target substance contained in the living sample derived from a patient at a clinical practice.

In the present patent application, Protein A, streptavidin, glucose oxidase, antibody and albumin may be excluded from the term “protein” used in the claims.

### REFERENTIAL SIGNS LIST

1: Gold substrate
2: Alkanethiol
3: Amino Acid
4: Protein

1. A method for immobilizing a protein on a self-assembled monolayer, the method comprising:
a step (a) of preparing a substrate comprising one molecule of an amino acid and the self-assembled monolayer, wherein
the one molecule of the amino acid is bound to the self-assembled monolayer through a peptide bond represented by the following chemical formula (I): 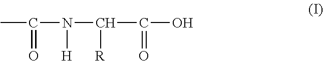
where R represents a side chain of the one molecule of the amino acid,
the one molecule of the amino acid is selected from the group of amino acids consisting of cysteine, lysine, histidine, phenylalanine, and glycine; and
a step (b) of supplying the protein to the substrate to form a peptide bond represented by the following chemical formula (II) as a result of reaction between a carboxyl group of the one molecule of the amino acid and an amino group of the protein: 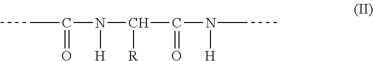
where R represents the side chain of the one molecule of the amino acid.

2. The method according to claim 1, wherein the step (a) comprises:
a step (a1) of preparing the substrate comprising the self-assembled monolayer on a surface thereof, the self-assembled monolayer having a carboxyl group at one end; and
a step (a2) of supplying the one molecule of the amino acid to form the peptide bond represented by the chemical formula (I) as a result of reaction between the carboxyl group of the one end of the self-assembled monolayer and an amino group of the one molecule of the amino acid.

3. The method according to claim 1, further comprising, between the step (a) and the step (b):
a step (ab) of activating a carboxyl group of the one molecule of the amino acid with a mixture of N-Hydroxysuccinimide and 1-ethyl-3-(3-dimethylaminopropyl) carbodiimide hydrochloride.

4. The method according to claim 2, further comprising, between the step (a1) and the step (a2):
a step (a1a) of activating the carboxyl group of the self-assembled monolayer with a mixture of N-Hydroxysuccinimide and 1-ethyl-3-(3-dimethylaminopropyl) carbodiimide hydrochloride.

5. The method according to claim 1, wherein the chemical formula (II) is represented by the following chemical formula (III): 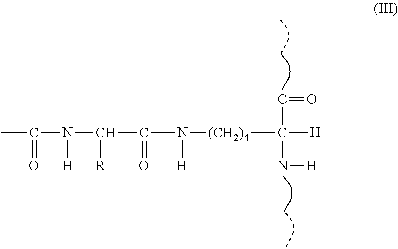
where R represents the side chain of the one molecule of the amino acid.

6. A sensor comprising:
a self-assembled monolayer;
one molecule of an amino acid; and
a protein, wherein:
the one molecule of the amino acid is interposed between the self-assembled monolayer and the protein,
the protein is bound to the self-assembled monolayer through two peptide bonds represented by the following chemical formula (II): 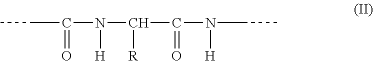
where R represents a side chain of the one molecule of the amino acid, and
the one molecule of the amino acid is selected from the group of amino acids consisting of cysteine, lysine, histidine, phenylalanine, and glycine.

7. The sensor according to claim 6, wherein the chemical formula (II) is represented by the following chemical formula (III): 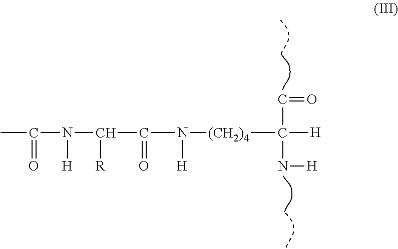
where R represents the side chain of the one molecule of the amino acid.

8. A method for detecting or quantifying a target substance contained in a sample with use of a sensor, the method comprising:
a step (a) of preparing the sensor comprising a self-assembled monolayer, one molecule of an amino acid, and a protein, wherein
the one molecule of the amino acid is interposed between the self-assembled monolayer and the protein,
the protein is bound to the self-assembled monolayer through two peptide bonds represented by the following chemical formula (II): 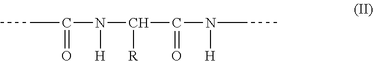
where R represents a side chain of the one molecule of the amino acid, and
the one molecule of the amino acid is selected from the group of amino acids consisting of cysteine, lysine, histidine, phenylalanine, and glycine;
a step (b) of supplying the sample to the sensor to bind the target substance to the protein; and
a step (c) of detecting the target substance bound in the step (b), or quantifying the target substance contained in the sample from an amount of the target substance bound in the step (b).

9. The method according to claim 8, wherein the chemical formula (II) is represented by the following chemical formula (III): 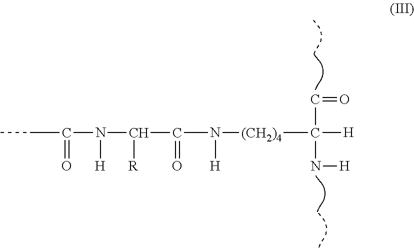
where R represents the side chain of the one molecule of the amino acid.

10. The method according to claim 1, wherein the protein does not include Protein A, streptavidin, glucose oxidase, antibody or albumin.

11. The sensor according to claim 6, wherein the protein does not include Protein A, streptavidin, glucose oxidase, antibody or albumin.

12. The method according to claim 8, wherein the protein does not include Protein A, streptavidin, glucose oxidase, antibody or albumin.
